(19) United States
(12) Patent Application Publication
(10) Pub. No.: US 2024/0351042 A1
(43) Pub. Date: Oct. 24, 2024

(54) OUTLET VALVE FOR A CONTAINER

(71) Applicant: Oribiotech LTD, Greater London, London (GB)

(72) Inventors: Farlan Veraitch, London (GB); William Raimes, London (GB); Enas Hassan, London (GB); Arman Amini, London (GB); Jason Palmer, Cambridge (GB)

(21) Appl. No.: 18/681,272

(22) PCT Filed: Aug. 2, 2022
(86) PCT No.: PCT/GB2022/052030

§ 371 Date: Feb. 5, 2024

(30) Foreign Application Priority Data
Aug. 5, 2021 (GB) 2111331.1

(51) Int. Cl. B01L 3/00; A61J 1/20; F16K 7/12

(52) U.S. Cl. CPC B01L 3/567; A61J 1/201; F16K 7/12

(57) ABSTRACT

There is described an outlet valve for a container, for example, a bioreactor container. The outlet valve comprises a membrane and the outlet valve is attachable to a wall of the container such that the membrane is arranged to face an internal volume of the container. The outlet valve further comprises an actuator operable to move membrane from: a first position in which the membrane is substantially in a plane of the wall of the container, to a second position in which a part of the membrane is displaced from the plane of the wall of the container such that the membrane is frustum-shaped.

### CROSS REFERENCE TO RELATED APPLICATIONS

This application is a national phase entry under 35 U.S.C. § 371 of International Patent Application PCT/GB2022/052030, filed Aug. 2, 2022, designating the United States of America and published as International Patent Publication WO 2023/012469 A1 on Feb. 9, 2023, which claims the benefit under Article 8 of the Patent Cooperation Treaty to British Patent Application Serial No. 2111331.1, filed Aug. 5, 2021.

### TECHNICAL FIELD

The disclosure relates to an outlet valve for a container, and preferably to a harvesting valve for a bioreactor container.

BACKGROUND

Cell and gene therapy manufacturing processes are often complex and include manual or semi-automated steps across several devices. Equipment systems used in various steps (i.e., unit operations) of cell-based therapeutic products (CTP) manufacturing may include devices for cell collection, cell isolation/selection, cell expansion, cell washing and volume reduction, cell storage and transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures, for example, but not limited to stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example, in an appropriate culture medium when culturing the cells. The known devices include shaker flasks, roller bottles, T-flasks and bags. Such bottles or flasks are widely used but suffer from several drawbacks. Chief among the problems are the requirement for transfer of cells without contamination when passaging or processing subsequently and the sterile addition of supplements and factors. The existing cell culture devices require re-supply of culture medium and oxygen for continued cell growth. Gas permeable cell culture devices are described in U.S. Pat. No. 8,415,144. However, such devices also require transfer of medium and/or cells in and out of the devices.

A key limiting factor in the production of cells or gene therapies for use in medicine is the absence of compact, automated closed systems for performing unit operations without contamination. For example, during cell culture, upstream or subsequent processing of cells, there is a risk of contamination when making additions to the culture vessel, or when removing cells or removing liquid samples. The operating systems are largely manual and hence expensive to operate. Multiple pieces of equipment are typically required to cover all of the non-cell culture steps, which involves many transfers, each of which is an opportunity for operator errors and contamination to occur. Furthermore, with increasing manual operations comes increasing risk of manual errors and therefore the current labor-intensive processes lack the robustness required for the manufacture of clinical-grade therapeutics.

Furthermore, to harvest cells following the cell culture process, the cell suspension is moved to an external container by transferring the cell suspension through an opening, such as by pouring the cell suspension, or by introducing laboratory instruments to extract the cell suspension, for example, using pipettes. This exposes the cell suspension and provides a risk of contamination, and such handling of the cell suspension may also damage the cells.

Accordingly, there is a need to improve harvesting of a cell suspension.

### BRIEF SUMMARY

It is an object of certain aspects of the present disclosure to provide an improvement over the above described techniques and known art; particularly to provide an improved arrangement to harvest the contents of a container, for example, a bioreactor container, which reduces, or eliminates, exposure of the contents to contaminants.

In accordance with one aspect of the present disclosure, there is provided an outlet valve for a container, for example, a bioreactor container, the outlet valve comprising a membrane and the outlet valve being attachable to a wall of the container such that the membrane is arranged to face an internal volume of the container, wherein the outlet valve further comprises an actuator operable to move the membrane from: a first position in which the membrane is substantially in a plane of the wall of the container, and a second position in which a part of the membrane is displaced from the plane of the wall of the container such that the membrane is frustum-shaped.

Accordingly, the outlet valve provides a simple and sterile means for extracting fluid from the bioreactor. The membrane provides a seal so that the contents of the bioreactor remain sterile during cell culture processes. In the first position the membrane is substantially in the plane of the wall of the container to provide a level surface for cell processing. The membrane can be moved to the second position in which it is frustrum-shaped to guide the contents of the container toward an outlet of the outlet valve.

In some examples, the outlet valve may alternatively be used as an inlet valve, for adding material to the container.

The outlet valve may further comprise an outlet conduit arranged such that the outlet conduit is sealed from the container by the membrane in the first position, and such that a fluid path may be formed between the container and the outlet conduit when the membrane is in the second position. The outlet conduit may provide a fluid pathway for the contents of the bioreactor container to flow out of an internal volume of the container.

In the second position the membrane is frustum-shaped. The frustum-shaped membrane may converge toward the outlet conduit. The frustum-shape of the valve in the second position helps to funnel the contents of the bioreactor toward the outlet conduit.

In the second position the membrane may be frusto-conical, or frusto-pyramidal, or other frustum-shaped. The outlet conduit may be disposed central to the membrane. The outlet conduit may alternatively be disposed at a position that is not central to the membrane.

The outlet valve may contain at least part of a needle arranged to pierce the membrane as the membrane is moved from the first position to the second position. The needle may be in fluid communication with the outlet conduit such that in the second position the contents of the container can flow through the needle and into the outlet conduit.

In an alternative example, the membrane may comprise an opening, for example, a slit or pinpoint opening, which is sealed in the first position and pulled open in the second position to create a fluid path through the membrane. In some examples, the outlet valve, in particular the outlet conduit, may comprise a protrusion that engages or aligns with the opening in the membrane in the second position.

In examples, the outlet valve may further comprise a housing that is attachable to the wall of the container. The housing may comprise the actuator. In some examples, the membrane is attached to the housing, and the housing is attachable to the wall of the container. In other examples, the membrane is attachable directly to the wall of the container and the housing is attachable to the wall of the container to house the membrane. The housing and/or membrane provide a closed, sterile environment in which cell culture processes can be carried out. The outlet valve and/or membrane may be hermetically sealed to the wall of the container.

The membrane may comprise a carrier portion connected to the part of the membrane that is displaced in the second position. The actuator may be arranged to engage the carrier portion such that operation of the actuator moves the carrier portion and moves the membrane to the second position.

The carrier part may have an opening to accommodate the needle or a part of the outlet conduit, for example, a protrusion of the outlet conduit. The carrier part may thereby control the direction of approach of the needle toward the membrane as the membrane is moved from the first position to the second position, or may control the alignment of the outlet conduit and an opening in the membrane.

The outlet valve may further comprise a needle hub arranged to hold the needle. The needle hub may be arranged to engage the carrier portion to guide the needle toward the membrane when the membrane is moved from the first position to the second position.

In examples, the actuator may be rotatable. The actuator may be rotatably mounted to the housing of the outlet valve. In other examples, the actuator may alternatively be slidable, pressable, or pull-able in order to move the membrane and/or the carrier portion from the first position to the second position.

In some examples, the actuator is rotatable and one of the actuator and the carrier portion comprises a cam, and the other of the actuator and the carrier portion comprises a cam follower arranged to engage the cam such that rotation of the actuator moves the carrier portion to move the membrane from the first position to the second position.

The cam may be configured to move the membrane from the first position to the second position by rotation of the moveable part by up to 360 degrees. The moveable part may comprise one or more gripping portions. The one or more gripping portions may be one or more lugs.

In examples, the outlet valve may comprise a lock arranged to prevent the membrane from being moved from the second position to the first position. This will prevent movement of the membrane back to the first position during or after harvesting of the contents of the bioreactor container. The lock can ensure that the outlet valve is ‘single use.’

The outlet valve may further comprise a gaiter arranged to extend between the membrane and a part of the housing such that outlet conduit and/or the needle is surrounded when the membrane is in the first position. One end of the gaiter may be attached to the membrane or the carrier portion. The gaiter maintains sterility of the fluid path between the internal volume of the container and the outlet conduit. The gaiter may be flexible, for example, compressible or collapsible, to accommodate movement of the membrane from the first position to the second position.

In accordance with another aspect of the present disclosure, there is provided a container, for example, a bioreactor container, comprising a plurality of walls defining an internal volume, and the outlet valve as described above mounted in a wall of the container such that the membrane faces the internal volume of the container.

The container may comprise a bottom wall and at least one side wall, and wherein the outlet valve may be mounted in the bottom wall of the container. This allows for harvesting of the contents of the container by gravity.

The container may further comprise a top wall or a lid. The top wall or lid may be an interface plate comprising one or more ports. The top wall or lid may seal the internal volume to provide a sterile environment for cell culture processes. The interface plate may provide for introduction or extraction of materials through the ports during the cell culture process.

In alternative examples, the outlet valve may be an inlet valve and may be mounted in the interface plate to provide for adding material into the container.

The side wall may be compressible. The side wall may have a compressible bellows structure. Accordingly, the bottom wall may be moveable toward the top wall or lid for mixing of the contents of the bioreactor and for adjustment of the volume of the bioreactor and the level of the contents of the container.

The bottom wall may be substantially circular. The bottom wall may be substantially planar.

The outlet valve may be offset from a center of the bottom wall. This provides for improved removal of low volumes of a fluid or cell suspension as container can be tilted and the contents removed by gravity. The outlet valve may alternatively be positioned at a central portion of the bottom wall.

The container may comprise a plurality of outlet valves. At least one of the outlet valves may be the outlet valve as described in the preceding paragraphs.

The container may further comprise a transfer conduit arranged to be in fluid communication with the container when the membrane is in the second position to transfer fluid out of the internal volume of the container. The transfer conduit may be a flexible tube. This allows for transfer of the harvested contents of the container to a patient, or to an external container such as a bag or a further bioreactor.

The flexible tube may be attached to the outlet valve, and wherein when the membrane is in the first position the flexible tube may be attached to the container for storage. The flexible tube may be stored by clipping the flexible tube to the bottom wall of the container. A cover may be detachably connected to the bottom wall. The cover may be hingedly connected to the bottom wall. The cover may cover the flexible tube and the outlet valve when not in use.

The container may further comprise a further container arranged to receive fluid from the container via the transfer conduit. The further container may be a bag. The bag may be attached to the container for storage. The further container may be a second bioreactor so that further cell culture processes can be carried out in the second bioreactor.

The container may be a bioreactor container for a cell culturing process.

The outlet valve may be a harvesting valve for harvesting cells after a cell culturing process has been performed in the bioreactor container.

### BRIEF DESCRIPTION OF THE DRAWINGS

Example embodiments of the disclosure are now described, by way of example only, hereinafter with reference to the accompanying drawings, in which:

FIG. 1 illustrates a perspective view of a bioreactor with a harvesting valve.

FIG. 2 illustrates a perspective view of a base wall of the bioreactor container.

FIG. 3 illustrates a side view of the bioreactor container and a sensor instrument.

FIG. 4 illustrates a perspective view of the base wall of the bioreactor container covered by a base cover.

FIG. 5 illustrates a side view of the bioreactor container and the base cover.

FIG. 6 illustrates a perspective view of the bioreactor assembled on a harvesting frame.

FIG. 7 illustrates a perspective view of the bioreactor assembled on a harvesting frame.

FIG. 8 illustrates a cross-sectional perspective view of the bioreactor container.

FIG. 9 illustrates an exploded view of the harvesting valve.

FIG. 10 illustrates a cross-sectional view of the harvesting valve.

FIG. 11(a) illustrates a bottom view of the harvesting valve in a first position.

FIG. 11(b) illustrates a bottom view of the harvesting valve in a second position.

FIG. 12(a) illustrates a perspective view of an internal side of the base wall of the bioreactor container including the harvesting valve in the first position.

FIG. 12(b) illustrates a perspective view of an internal side of the base wall of the bioreactor container including the harvesting valve in the second position.

FIG. 13(a) illustrates a cross-sectional view of the harvesting valve in the first position.

FIG. 13(b) illustrates a cross-sectional view of the harvesting valve in the second position.

### DETAILED DESCRIPTION

The described example embodiments relate to an assembly for handling biological material. In particular, some embodiments relate to an assembly that is aseptic, or sterile. It is noted that the terms “aseptic” and “sterile” may be used interchangeably throughout the present disclosure. References to fluids in the detailed description are not intended to limit the scope of protection to such materials. As will be recognized by a person skilled in the art, fluids as described herein are merely an example of a suitable material for use with the assembly as described. Equally, reference may be made to a container, bioreactor, or the like, however, such references are not intended to limit the scope of protection to such containers or bioreactors. As will be recognized by a person skilled in the art, containers, bioreactors or the like are described herein as mere examples.

Certain terminology is used in the following description for convenience only and is not limiting. The words ‘upper’ and ‘lower’ designate directions in the drawings to which reference is made and are with respect to the described component when assembled and mounted. The words ‘inner,’ ‘inwardly’ and ‘outer,’ and ‘outwardly’ refer to directions toward and away from, respectively, a designated centerline or a geometric center of an element being described (e.g., a central axis), the particular meaning being readily apparent from the context of the description. Further, the terms ‘proximal’ (i.e., nearer to) and ‘distal’ (i.e., away from) designate positions relative to an axis or a point of attachment.

Further, as used herein, the terms ‘connected,’ ‘affixed,’ ‘coupled’ and the like are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first,’ ‘second,’ ‘third’ etc., merely indicate that different instances of like objects are being referred to and are not intended to imply that the objects so described must be in a given sequence, either temporally, spatially, in ranking or in any other manner. Like reference numerals are used to depict like features throughout.

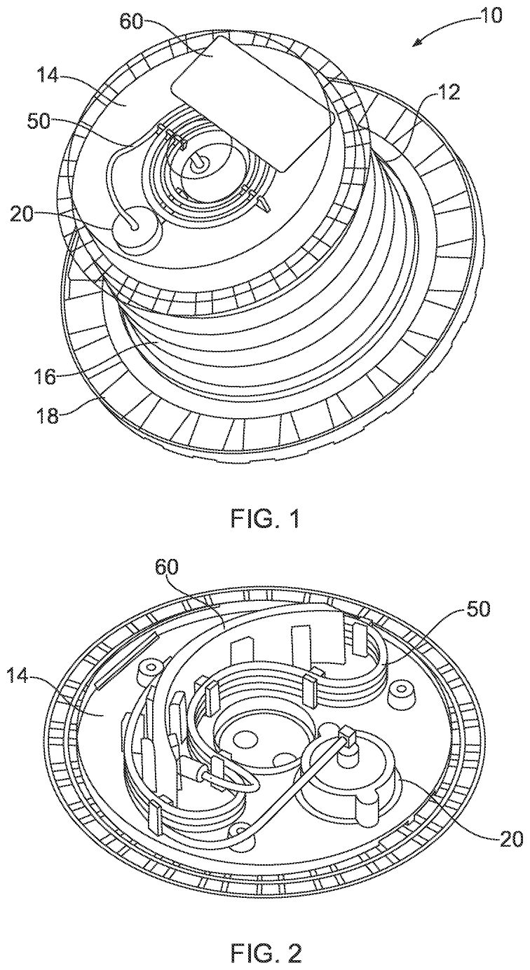

FIG. 1 shows a bioreactor 10 including a container 12 having a base wall 14 and a side wall 16 having an internal volume therebetween. The internal volume is adapted to hold a cell suspension. The base wall 14 is circular. However, the base wall 14 may be any other suitable shape, such as oval, square, triangular, or another polygonal shape. The base wall 14 may be substantially planar. The container 12 may be expandable along a longitudinal axis thereof. For example, the side wall 16 may be compressible. The compressible side wall 16 may have a bellows structure. The bellows structure has a plurality of lateral rigid sections. Each pair of adjacent lateral rigid sections is interleaved with a deformable region so as to allow compression of the bioreactor along the longitudinal axis.

The bioreactor 10 is also provided with an interface plate 18 connected to the side wall 16 such that the interface plate 18 seals the internal volume of the container 12. The interface plate 18 is opposite to the base wall 14. The interface plate may include a plurality of ports through which materials, such as fluids or solid suspensions, can be added to and/or extracted from the internal volume. The materials may include cells, culture media, growth factors, proteins, samples, washing solutions, beads, antibodies, viruses, or any other materials used for cell culture. During the cell culture process, such materials can be added or extracted through the ports at pre-determined times or as and when required. The bioreactor 10 can thus be used to culture cells in a closed system.

To culture cells in the bioreactor 10, cells are added into the container 12. The cells may be added in a suspension. Culture media is added to the container together with the cells, prior to adding the cells or following addition of the cells. Other materials may also be added to the container, for example, growth factors, proteins, washing solutions, beads, antibodies or viruses. The cells can proliferate and expand in the container. The cells may be washed by removing waste media, optionally using a buffer to wash the cells, and then adding new culture media to the bioreactor. At the end of the cell culture process, the cell suspension is removed from the container, this is known as cell harvesting. Once the cells are harvested, the cells may transferred to another container 60, for example, a further bioreactor or a bag. The harvested cells may be injected into a patient.

FIGS. 1 to 3 show an outlet valve, specifically a harvesting valve 20, provided in the base wall 14 of the container 12. The harvesting valve 20 may be connected to a transfer tube 50 that can be used to connect the container 12 to a further container 60. The transfer tube 50 may be a flexible tube. The further container may be a bellows container as described herein, a bag 60 or any other suitable sterile container. Initially, the harvesting valve 20 is sealed. The harvesting valve 20 is operable to open a fluid path between the container 12, the transfer tube 50 and the further container 60 for moving cells from the container 12 to the further container 60.

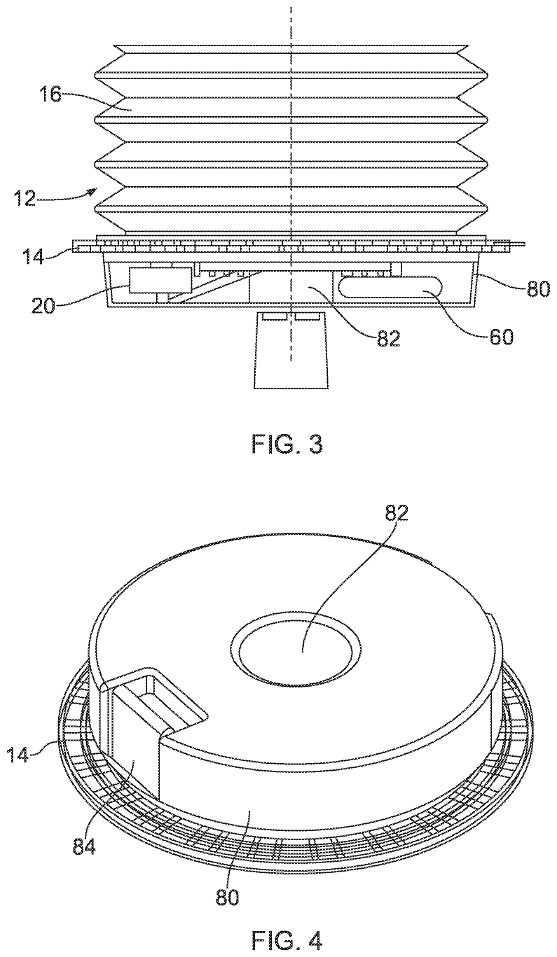

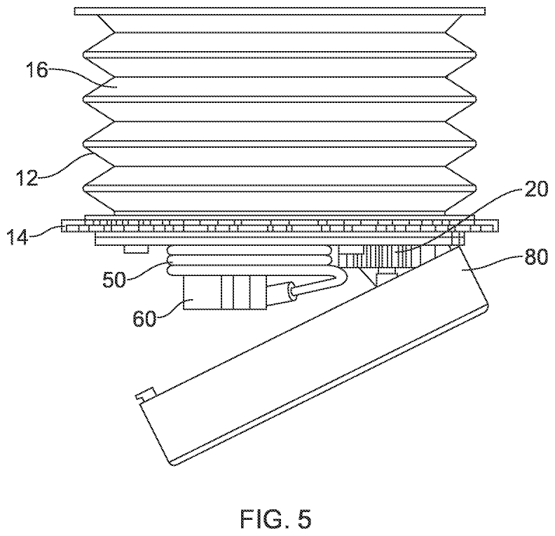

The harvesting valve 20 and the transfer tube 50 may be provided preassembled with the bioreactor 10. The bag 60 may also be provided preassembled to the transfer tube 50. The base wall 14 may include clips on a lower side thereof, opposite the internal volume of the container 12, to hold the transfer tube 50 and/or the bag 60. A base cover 80, as shown in FIGS. 3 to 5, may be provided to cover the harvesting valve 20, transfer tube 50 and bag 60 when not in use to protect the bag 60 and to prevent unintentional actuation of the harvesting valve 20. The base cover 80 may include an aperture 82 that permits instruments, for example, sensing instruments, to contact or otherwise interact with the base wall 14. The base cover 80 may be hingedly connected, or otherwise removably connected to the base wall 14. The base cover 80 may further comprise a clip 84 to connect the base cover 80 to the base. A user or an automated device can operate the clip 84 to remove the base cover 80 to access the harvesting valve 20. The base cover 80 is removed when the contents of the bioreactor 10 are ready to be harvested.

The bioreactor 10, the harvesting valve 20, the transfer tube 50 and optionally the bag 60 may be pre-assembled in a sterile environment or sterilized prior to use to provide a sealed, sterile connection between the bioreactor 10 and the transfer tube 50. According to an alternative arrangement, a transfer tube 50 can be connected to the harvesting valve 20 by a user when the cells are ready to be harvested. The transfer tube 50 may be connected to the harvesting valve 20 in a sterile environment, such as a laminar flow hood or a clean room, to avoid contamination of the harvested cells.

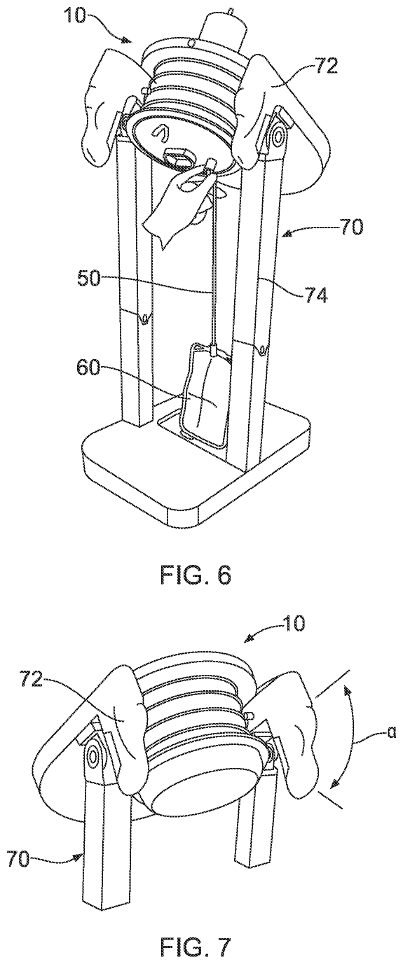

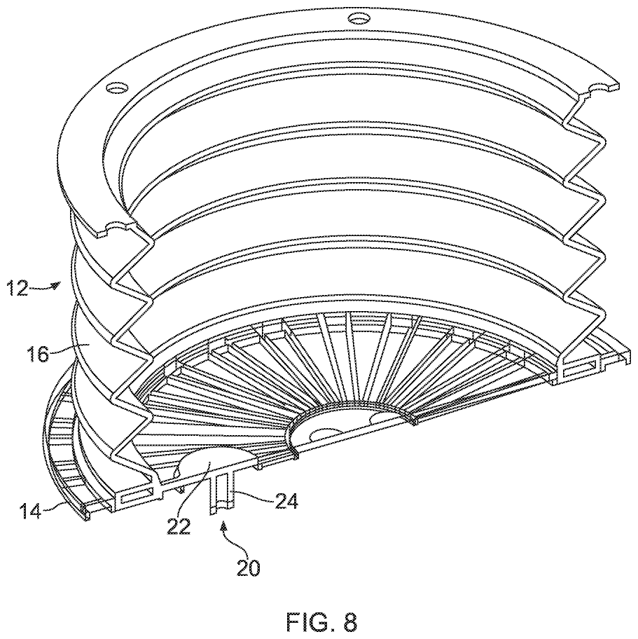

The harvesting valve 20 has a membrane 22 that is coupled to the base wall 14, as shown in FIG. 8. The membrane 22 faces the internal volume of the container 12. The membrane 22 may be provided at a position offset from a center of the base wall 14. As will become apparent hereinafter, providing the membrane 22 in this position allows for improved removal of the contents of the container 12 when harvesting, particularly when harvesting a low volume of cell suspension, as the bioreactor 10 can be tilted toward the membrane 22 of the harvesting valve 20 as shown in FIGS. 6 and 7. The membrane 22 may alternatively be positioned in the center of the base wall 14. The membrane 22 may be circular. The membrane 22 may alternatively be any other suitable shape, for example, oval, square, triangular, or other polygonal shape. The membrane 22 is formed from a flexible material. The flexible material may be an elastic material. The flexible material may be a thermoplastic elastomer (TPE), silicone, low density polyethylene (LDPE), or any other suitable flexible or elastic material. The membrane 22 may be hermetically sealed to the base wall 14. The hermetic seal may be formed by integrally molding or co-extruding the membrane 22 with the base wall 14, or by welding the membrane 22 to the base wall 14, for example, ultrasonically welding. In other examples, as described hereinafter, the membrane 22 may be sealingly attached to a housing, which is in turn sealingly attached to the base wall 14 such that the membrane 22 faces the internal volume of the container 12 and the base wall 14 is sealed.

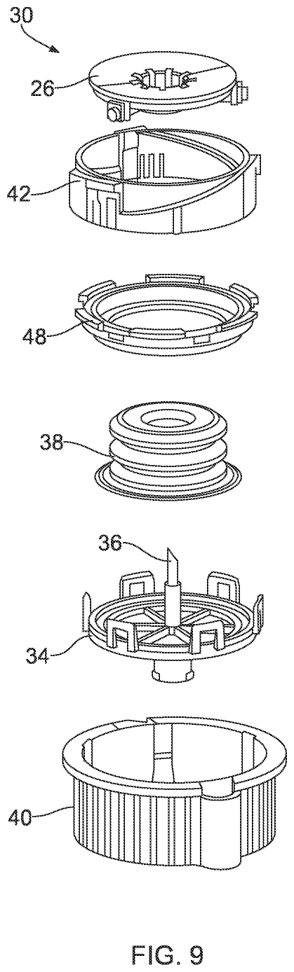

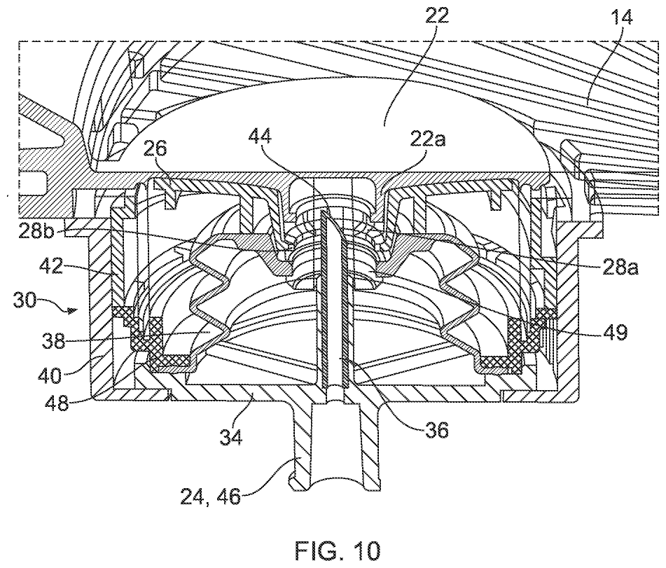

The harvesting valve 20 also has a housing 30, as shown in FIG. 9, including an actuator 32, a needle hub 34, a needle 36 fixedly mounted to the needle hub 34, a gaiter 38 and a cam cap 48. The actuator 32 has a rotatable part 40 and a cam ring 42 having a cam surface. As shown in FIG. 10, the housing 30 is connected to the base wall 14 immediately surrounding the membrane 22 on a surface opposite to the internal volume of the container 12. The housing 30 may alternatively be connected to the membrane 22 on a surface opposite to the internal volume of the container 12.

According to an alternative arrangement, the membrane 22 may be part of the housing 30. The housing 30 may include the membrane 22 that is attached to, for example, press-fitted into or welded to, the base wall 14 to provide a fluid-tight seal.

As shown in FIG. 8, an outlet conduit 24 extends from the harvesting valve 20, away from the internal volume of the container 12. The outlet conduit 24 may be integrally formed with the membrane 22 or molded or otherwise attached thereon, as described further hereinafter. The outlet conduit 24 may be positioned at a central part of the membrane 22. Prior to actuation of the harvesting valve 20, the outlet conduit 24 is sealed from the container 12 by the membrane 22.

As shown in FIG. 10, the membrane 22 includes a molded connection formation 22a extending away from the internal volume of the container 12 and defining a pierceable area. The molded connection formation 22a may be integrally formed with the membrane 22, molded thereon, or attached thereto. The connection formation 22a may be positioned at a central part of the membrane 22. The molded connection formation 22a may be formed from the same material as the membrane 22.

A carrier portion 26 is connected to the membrane 22, in particular the connection formation 22a. As illustrated in FIG. 10, a plug 49 is arranged to connect the molded connection formation 22a to the carrier portion 26. The plug 49 is integrally molded with, or attached to, the connection formation 22a. The plug 49 may be formed of a rigid material, for example, polycarbonate, high-density polyethylene (HDPE), or any other suitable rigid polymers. As shown in FIG. 10, the carrier portion 26 is provided with a circumferential lip 28a and the plug 49 is provided with a circumferential groove 28b so that the circumferential lip 28a and the circumferential groove 28b interconnect to provide a secure connection between the connection formation 22a and the carrier portion 26 via the plug 49.

Alternatively, the carrier portion 26 may be connected to the plug 49 or the connection formation 22a by a clamping arrangement or a threaded connection, or the carrier portion 26 may be connected to the plug 49 or membrane 22 by an adhesive or by corresponding attachment formations. In examples, no plug 49 is provided and the carrier portion 26 is attached directly to the membrane 22. In examples, the membrane 22 does not include the connection formation 22a and the carrier portion 26 is attached to the membrane 22 by adhesive, or welding, or the like.

The carrier portion 26 has a cam follower and is connectable to the cam ring 42 of the actuator 32. Rotation of the actuator 32 will cause translational movement of the carrier portion 26 to move the membrane 22 from a first position to a second position. The details of the actuator are described in further detail below. The translational movement of the carrier portion 26 is in a direction normal to the plane of the base wall 14.

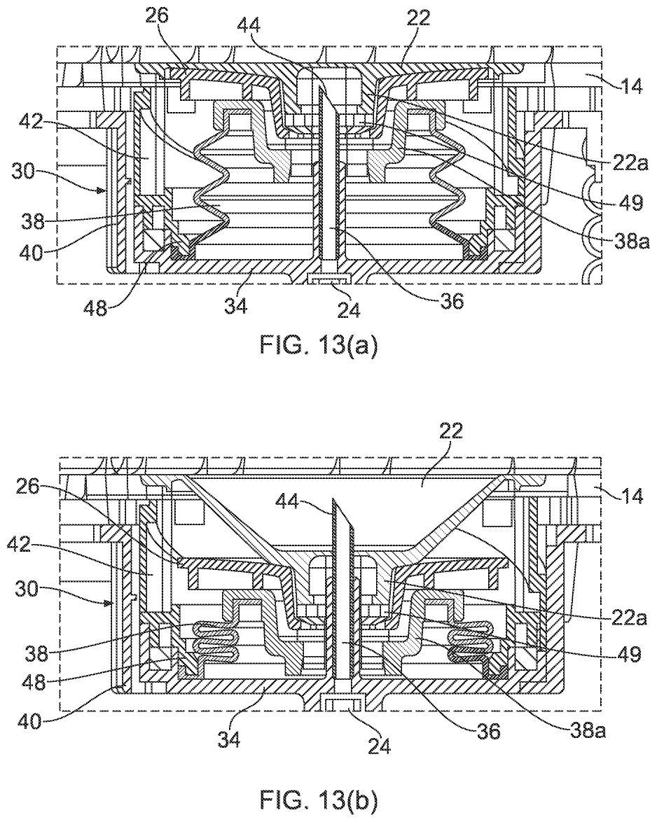

In the first position, shown in FIGS. 12a and 13a, the membrane 22 is substantially in the plane of the base wall 14 of the container 12. The membrane 22 is substantially flat when in the first position. Alternatively, the membrane 22 may be curved when in the first position, for example, to correspond to the surface of a curved base wall 14.

The actuator 32 is operable to displace a part of the membrane 22 from the plane of the base wall 14 of the container 12 to the second position, shown in FIGS. 12b and 13b, in which the membrane is frustum-shaped. The displaced part of the membrane 22 may be a central part of the membrane 22. Alternatively, the displaced part may be offset from a center of the membrane 22. In the second position, the frustum-shaped membrane 22 converges toward the outlet conduit 24.

As the membrane 22 is moved to the second position, an opening is formed in the membrane. The opening may be formed by piercing the membrane with the needle 36 as the membrane 22 moves toward the second position. The needle 36 may be held stationary by the needle hub 34 so that translation of the membrane 22 by the actuator 32 pulls the membrane 22 toward the needle 36, which pierces the membrane to create the opening. A fluid path is thereby created between the internal volume of the container 12 and an internal conduit of the needle 36. The contents of the bioreactor 10 will be directed toward a bevel 44 of the needle 36 by the converging frustum-shaped membrane 22 and can thus be harvested.

According to an alternative arrangement, the needle 36 may be actuated, for example, pushed or slid, to pierce the membrane 22 after the membrane 22 has been moved to the second position.

The needle 36 is preferably a large gauge needle to maximize the harvest yield and flow rate. As a consequence, the membrane 22 is not re-sealable following harvesting of the cell suspension. The container 12 is thus a single-use container and cannot be re-used following harvesting of the cell suspension.

According to an alternative arrangement, a smaller needle may be used to pierce the membrane 22. The membrane 22 may also be a self-sealing polymer, for example, silicone. Accordingly, the membrane 22 maintains a seal after being pierced. The membrane 22 may be pierced more than once and continue to maintain a seal. This allows the valve 20 to be used for multiple harvests of the cell solution, or to take one or more samples of the cell solution for testing. This arrangement may alternatively be used as an inlet valve, for adding material to the container.

The plug 49 and/or the molded connection formation 22a may accommodate the bevel end 44 of the needle 36 so that the needle 36 can pass through and pierce the membrane 22 as the membrane 22 is moved from the first position to the second position. In particular, the plug 49 and/or the molded connection formation 22a may have an opening through which the needle 36 passes. The opening in the plug 49 and/or molded connection formation 22a may cooperate with the needle 36 and/or the needle hub 34 to guide the movement and improve stability between the needle 36, the membrane 22 and the carrier portion 26. This can control the direction of approach of the needle 36 toward the membrane 22.

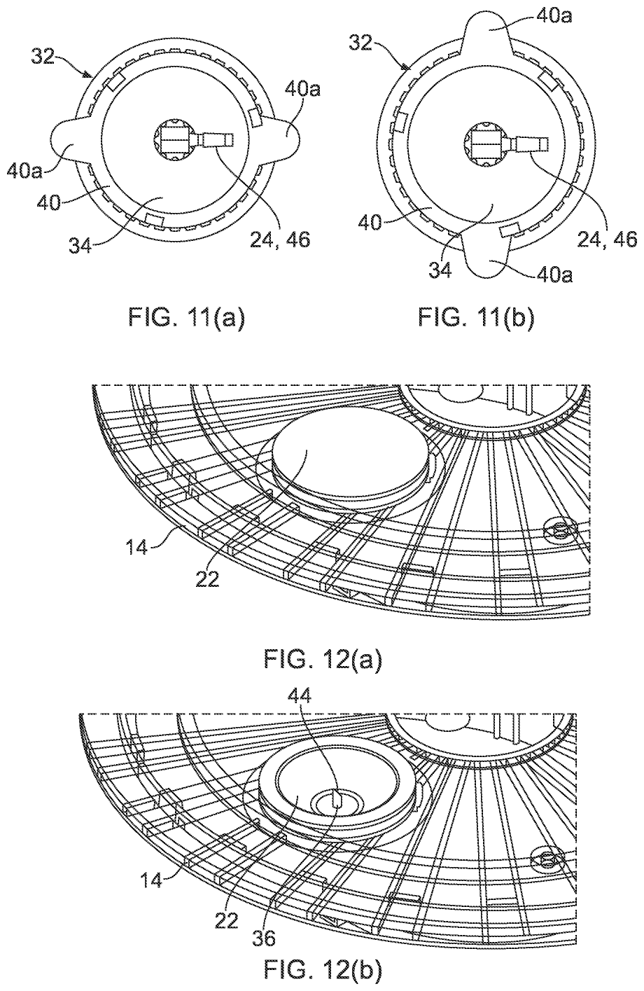

The rotatable part 40 of the actuator 32 is fixedly coupled to the cam ring 42. Accordingly, rotation of the rotatable part 40 also rotates the cam ring 42. The rotatable part 40 may have one or more gripping features, for example, lugs 40a as shown in FIGS. 11(a) and 11(b), to allow a user to grip and rotate the rotatable part 40.

The needle hub 34 may be mounted to the rotatable part 40, as illustrated, or to the cam ring 42. The needle hub 34 may be rotatably coupled to the rotatable part 40 such that rotation of the rotatable part 40 does not rotate the needle hub 34 or needle 36.

As shown in FIG. 10, the gaiter 38 is clamped at a lower end between the cam cap 48 and the needle hub 34 and extends upwards to the carrier portion 26 where it is attached to the carrier portion, for example, by a circlip or similar. The gaiter 38 may be rigidly connected to the needle hub 34 and the carrier portion 26. The carrier portion 26 may be rotatably restrained to the cam cap 48, the needle hub 34 and/or the gaiter 38, and arranged such that the carrier portion 26 is able to slide axially relative to the housing 30.

Rotation of the rotatable part 40 rotates the cam ring 42. As the cam ring 42 rotates, the cam follower of the carrier portion 26 interacts with the cam on the cam ring 42 to translate the carrier portion 26 in a direction normal to the plane of the base wall 14, thereby moving the membrane 22 from the first position to the second position.

To achieve translation of the membrane 22 from the first position to the second position, the rotatable part may be rotated by a quarter turn (i.e., about 90 degrees), a half turn (i.e., about 180 degrees), a full turn (i.e., about 360 degrees), or any other suitable degree of rotation.

As shown in FIG. 11(a), the rotatable part 40 is in the closed position. To actuate the valve 20 and move the membrane 22 to the second position, the rotatable part may be turned 90 degrees to the position shown in FIG. 11(b).

According to an alternative arrangement, the rotatable part 40 of the actuator 32 comprises a cam surface that is coupled to the cam follower of the carrier portion 26. Rotation of the rotatable part will therefore cause the cam follower to interact with the cam on the rotatable part 40 to translate the carrier portion 26 in a direction normal to the plane of the base wall 14, thereby moving the membrane 22 from the first position to the second position.

In an alternative arrangement, the carrier portion 26 may have a cam surface, and the rotatable part 40 or cam ring 42 may have a cam follower arranged to engage the cam surface on the carrier portion 26. Accordingly, rotation of the rotatable part 40 and/or cam ring 42 would cause translation of the carrier portion 26 in the same manner as described above.

As shown in FIGS. 10 and 13(a), the gaiter 38, extends between the needle hub 34 and the carrier portion 26 and surrounds the needle 36. The gaiter 38 maintains sterility of the fluid path between the internal volume of the container 12 and the outlet conduit 24, in this example a valve outlet 46. The gaiter 38 has an end plate 38a that is connected to the carrier portion 26 and/or to the molded connection formation 22a of the membrane 22. The gaiter 38 has a collapsible concertina wall. The concertina wall provides a collapsible wall that has sufficient rigidity to support the carrier portion 26 when the membrane 22 is in the first position and sufficient flexibility to collapse as the membrane 22 and carrier portion 26 are actuated to move toward the second position.

The housing 30 may include a locking arrangement (not shown) to lock the membrane 22 and/or the carrier portion 26 in the second position. This will prevent movement of the membrane 22 to the first position during harvesting of the contents of the bioreactor 10. For example, the locking arrangement may be a leaf spring in the housing 30 (e.g., in the cam ring 42), which engages a corresponding opening in the carrier portion 26 to lock the carrier portion 26 in the second position.

In examples, the outlet conduit 24 of the harvesting valve 20 comprises a valve outlet 46 formed in the housing 30 and in communication with the needle 36. The valve outlet 46 is formed in the needle hub 34 and is fluidly connected to the internal conduit of the needle 36. The valve outlet 46 may be a luer interface or a barbed spigot. The valve outlet 46 can be connected to the transfer tube 50. Alternatively, the bioreactor 10 may be provided with the transfer tube 50 preassembled to the valve outlet 46, thereby providing a sterile connection between the transfer tube 50 and the harvesting valve 20.

To harvest the contents of the bioreactor 10, the bioreactor may be placed on support arms 72 of a harvesting frame 70, as shown in FIGS. 6 and 7. The harvesting frame 70 further comprises a stand 74, the support arms 72 are pivotal with respect to the stand 74 by an angle α. The support arms may be pivotable from an angle α of approximately −60 degrees to approximately 60 degrees from a horizontal position. The support arms are preferably pivotable from an angle α of approximately −45 degrees to 45 degrees from the horizontal position.

The method of harvesting cells includes placing the bioreactor 10 on the support arms 72. The arms are pivoted to any suitable angle α so that the harvesting valve 20 is positioned toward the lowest point of the base wall 14. The arms may be pivoted to an angle α of 45 degrees from a horizontal position. A bag or further container 60 and the transfer tube 50 may be preassembled with the harvesting valve 20. Alternatively, transfer tube 50 and a bag or further container 60 can be connected to the harvesting valve 20 after it is placed on the harvesting frame 70. The further container 60 is placed at a position below the bioreactor 10. The harvesting valve 20 is then opened by a user, for example, by rotating the rotatable part 40 to move the membrane 22 from the first position to the second position. The membrane 22 may be pierced. A fluid pathway is thereby created through the harvesting valve 20, between the bioreactor 10 and the bag or further container 60. The contents of the bioreactor will then flow under gravity and transfer to the bag or further container 60. Once harvesting is complete, the bag or further container 60 can be sealed.

During harvesting of the cell suspension from the bioreactor 10, the valve 20 provides a two-way passage for fluid. This allows for fluids, such as a saline buffer or the harvested cell suspension, to be flushed into the internal volume of the container 12 during harvesting to wash the internal volume of the bioreactor 10 and maximize the harvest yield. According to an alternative arrangement, the valve 20 may only permit fluid to flow in a single direction from the internal volume of the container 12 to the transfer tube 50. For example, a one-way valve may be provided in the needle 36 or in the outlet conduit 24 to prevent backflow of the cell suspension into the internal volume.

According to an alternative arrangement, the bioreactor may be tilted by an actuation plate within an automated system. The harvesting valve 20 can then be actuated by the automated system to harvest the contents of the bioreactor 10.

According to an alternative embodiment, instead of the needle 36 that pierces the membrane 22, the membrane 22 may include a slit or a pinpoint opening. The slit or pinpoint opening may be sealed when the membrane is in the first position, and exposed or opened when the membrane is moved to the second position to create a fluid path between the internal volume of the container 12 and the outlet conduit 24.

The actuator 32 has been described herein as a rotatable part 40 and a cam ring 42, however alternative forms actuators are contemplated for use with the present disclosure. For example, the actuator may be slid, pressed or pulled in order to actuate the harvesting valve 20 to move the membrane 22 from the first position to the second position.

The harvesting valve 20 has been described herein as being connected to the base wall 14. The harvesting valve 20 may alternatively be connected to the side wall 16 or to the interface plate 18. The bioreactor 10 may be tilted and/or inverted to harvest the contents of the bioreactor 10 so that the harvesting valve 20 is positioned at a lower end thereof when harvesting the contents. The container 12 described herein has been described as having a single harvesting valve 20. However, the container 12 may be provided with a plurality, for example, two or more, harvesting valves 20.

Generally, it will be appreciated by persons skilled in the art that the above embodiments have been described by way of an example only and not in any limitative sense, and that various alternations and modifications are possible without departing from the scope of the disclosure as defined by the appended claims. Various modifications to the detailed designs as described above are possible, for example, variations may exist in shape, size, arrangement, assembly, sequence or the like. For example, any one of the enclosures, planar interfaces, component retaining elements or the like may be used in any suitable combination. Moreover, whilst the present disclosure has been described in relation to an automated process, it will be appreciated by persons skilled in the art that a user may manually, or semi-automatedly, undertake one or more of the above process steps.

1. An outlet valve for a container, comprising:
a membrane
arranged to face an internal volume of the container, the outlet valve attachable to a wall of the container; and
an actuator operable to move the membrane:
from a first position in which the membrane is substantially in a plane of the wall of the container;
to a second position in which a part of the membrane is displaced from the plane of the wall of the container, the membrane includes a frustum-shape in the second position.

2. The outlet valve of claim 1, further comprising an outlet conduit, wherein the membrane seals the outlet conduit from the container in the first position and opens a fluid path between the container and the outlet conduit in the second position.

3. The outlet valve of claim 2, wherein, in the second position, the frustum-shape of the membrane converges toward the outlet conduit.

4. The outlet valve of claim 2, wherein the outlet conduit is disposed central to the membrane.

5. The outlet valve of any of claim 2, further comprising a needle, at least a part of the needle arranged to pierce the membrane as the membrane is moved from the first position to the second position to open the fluid path.

6. The outlet valve of claim 1, further comprising a housing that is attachable to the wall of the container, the housing comprising the actuator.

7. The outlet valve of claim 1, further comprising a carrier portion connected to the part of the membrane that is displaced in the second position, wherein the actuator is arranged to engage the carrier portion and move the carrier portion causing the membrane to move from the first position to the second position.

8. The outlet valve of claim 7, wherein the actuator is rotatable.

9. The outlet valve of claim 8, wherein one of the actuator and the carrier portion comprises a cam, and the other of the actuator and the carrier portion comprises a cam follower arranged to engage the cam, wherein rotation of the actuator moves the carrier portion causing the membrane to move from the first position to the second position.

10. The outlet valve of claim 9, wherein the cam is configured to move the membrane from the first position to the second position by rotation of the actuator by up to 360 degrees.

11. The outlet valve of claim 1, wherein the actuator comprises one or more gripping portions.

12. The outlet valve of claim 1, comprising a lock arranged to prevent the membrane from being moved from the second position to the first position.

13. A container comprising:
a plurality of walls defining an internal volume; and
an outlet valve mounted to a wall of the container, the outlet valve comprising:
a membrane arranged to face the internal volume of the container; and
an actuator operable to move the membrane:
from a first position in which the membrane is substantially in a plane of the wall of the container;
to a second position in which a part of the membrane is displaced from the plane of the wall of the container, the membrane includes a frustum-shape in the second position.

14. The container of claim 13, wherein the plurality of walls comprises a bottom wall and at least one side wall, and wherein the outlet valve is mounted to the bottom wall of the container.

15. The container of claim 14, wherein the bottom wall is substantially circular and substantially planar.

16. The container of claim 14, wherein the outlet valve is offset from a center of the bottom wall.

17. The container of claim 13, comprising a plurality of outlet valves.

18. The container of claim 13, further comprising a transfer conduit arranged to be in fluid communication with the container when the membrane is in the second position to transfer fluid out of the internal volume of the container.

19. The container of claim 18, wherein the transfer conduit is a flexible tube.

20. The container of claim 19, wherein the flexible tube is attached to the outlet valve, and wherein when the membrane is in the first position the flexible tube is attached to the container for storage.

21. The container of claim 18, further comprising a further container arranged to receive fluid from the container via the transfer conduit.

22. The container of any of claim 13, wherein the container is a bioreactor container for a cell culturing process.

23. The container of claim 22, wherein the outlet valve is a harvesting valve for harvesting cells after a cell culturing process has been performed in the bioreactor container.
